(19) United States
(12) Patent Application Publication
(10) Pub. No.: US 2026/0199637 A1
(43) Pub. Date: Jul. 16, 2026

(54) HANDLE AND CATHETER SYSTEM WITH HANDLE

(71) Applicant: KANEKA CORPORATION, Osaka (JP)

(72) Inventors: Hirokazu Kamakura, Nagano (JP); Yuki Mukai, Nagano (JP)

(73) Assignee: KANEKA CORPORATION, Osaka (JP)

(21) Appl. No.: 19/559,027

(22) Filed: Mar. 6, 2026

Related U.S. Application Data
(63) Continuation of application No. PCT/JP2024/028434, filed on Aug. 8, 2024.

(30) Foreign Application Priority Data
Sep. 7, 2023 (JP) 2023-145185

(51) Int. Cl. A61M 25/06; A61M 37/00

(52) U.S. Cl. CPC A61M 25/0662; A61M 37/00; A61M 2202/0007; A61M 2202/04

(57) ABSTRACT

A handle having an inner cavity configured to receive a tube connected to a puncture needle is provided. The handle includes a contact member including a portion that is in contact with an outer surface of the tube. In a cross-section passing through a central axis of the tube and extending along a longitudinal direction of the tube, the outer surface of the tube includes a plurality of contact portions that are in contact with the contact member and a non-contact portion that is present between the contact portions and is not in contact with the contact member, and the puncture needle and the contact member are electrically connected to each other via the contact portions. A catheter system includes the handle.

### CROSS-REFERNCE TO RELATED APPLICATIONS

This application claims priority to Japanese Patent Application No. 2023-145185 filed on September 7, 2023, the entire contents of which are incorporated herein by reference.

### TECHNICAL FIELD

One or more embodiments of the present invention relate to a handle and a catheter system including the handle.

### BACKGROUND

Therapies and the like that involve directly administering a drug solution such as a myocardial regenerative cell preparation to cardiac muscle cells that are losing the functions thereof owing to a myocardial infarction or the like so as to regenerate the cardiac muscle cells are performed. In a case where such a drug solution is directly administered to an organ inside a body in this manner, a catheter having a needle needs to be inserted into a body cavity, and the needle needs to be caused to puncture the organ. In some cases, when puncturing the organ with the needle, it is necessary to transmit torque to the needle while supplying electricity to the needle in order to grasp the puncture depth of the needle. As a configuration for transmitting torque while supplying electricity to the needle, for example, the following one has been developed.

Patent Literature 1 describes a drug delivery catheter for injecting a small amount of a therapeutic agent into the heart wall, which includes: a catheter tube suitable for percutaneous insertion into the heart and having a distal end suitable for insertion into the heart and a proximal end housed in a housing; and a piercing needle suitable for piercing the heart wall and disposed at the distal tip of a first lumen. It is disclosed that this catheter has a conductive helical coil mechanically crimped to a pin by a crimp, and that rotation of the pin or crimp relative to a catheter body causes the coil to rotate, thereby rotating the piercing needle.

### PATENT LITERATURE

PTL 1: Japanese Laid-Open Patent Publication (Translation of PCT Application) No. 2003-529409

However, the configuration described in Patent Literature 1 for transmitting torque to the needle while supplying electricity still has room for improvement.

### SUMMARY

One or more embodiments of the present invention have been made in view of the above, and a novel handle that allows a tube disposed in an inner cavity of the handle to be easily moved and rotated while supplying electricity to a puncture needle, and a catheter system including the handle are provided.

A handle according to one or more embodiments of the present invention which can achieve the above object is as follows.

1. A handle having an inner cavity into which a tube connected to a proximal end portion of a medical puncture needle having a longitudinal direction is inserted, the tube having an inner cavity extending in the longitudinal direction, wherein

in a state where the tube is inserted into the inner cavity of the handle,

the handle comprises a contact member including a portion that is in contact with an outer surface of the tube,

in a cross-section passing through a central axis of the tube and extending along a longitudinal direction of the tube, the outer surface of the tube includes a plurality of contact portions that are in contact with the contact member, and a non-contact portion that is present between the contact portions and is not in contact with the contact member, and

the puncture needle and the contact member are electrically connected to each other via the contact portions.

Since the non-contact portion is present between the plurality of contact portions, it becomes easier to reduce the area of contact between the outer surface of the tube and the contact member. Therefore, it can be made easier to reduce frictional resistance generated between the outer surface of the tube and the contact member. Accordingly, it becomes easier to move the tube in the longitudinal direction of the puncture needle and to rotate the tube in the circumferential direction of the puncture needle within the handle. In addition, since the puncture needle and the contact member are electrically connected to each other via the contact portions, electricity can be supplied to the puncture needle. Therefore, the handle allows the tube disposed in the inner cavity of the handle to be easily moved and rotated while supplying electricity to the puncture needle.

The handle according to one or more embodiments of the present invention may be any of [2] to [7] below.

2. The handle according to above [1], wherein, in the cross-section passing through the central axis of the tube and extending along the longitudinal direction of the tube, a length of the contact portion in the longitudinal direction of the puncture needle is shorter than a length of the non-contact portion in the longitudinal direction of the puncture needle.

3. The handle according to above [1] or [2], wherein the contact member is a coil in which a wire is wound helically.

4. The handle according to above [3], wherein a pitch of the coil is larger than a wire diameter of the wire.

5. The handle according to above [1] or [2], wherein the contact member is a projecting member provided so as to project radially inward of the tube.

.6. The handle according to above [5], wherein

a plurality of the projecting members are provided, and

one projecting member is separated from another projecting member.

7. The handle according to any one of above [1] to [6], wherein the contact member is made of a conductive material.

One or more embodiments of the present invention also include the following catheter system.

8. A catheter system comprising:

the handle according to any one of above [1] to [7];

the puncture needle;

the tube to which the puncture needle is connected; and

a sheath connected at a proximal end portion thereof to the handle and having an inner cavity into which the puncture needle and the tube are inserted.

9. The catheter system according to above [8], wherein the puncture needle and the tube are made of a conductive material.

The catheter system according to above [8] or [9], wherein the puncture needle includes: a tubular member having an inner cavity extending in the longitudinal direction of the puncture needle; and a helical member in which a wire is wound helically around the tubular member.

The catheter system according to any one of above [8] to , wherein the tube includes a first tube having a plurality of grooves formed therein.

The catheter system according to above , wherein

the tube includes a second tube connected to a proximal end portion of the first tube, and

the contact member comes into contact with an outer surface of the second tube.

The catheter system according to above or , wherein a first resin layer made of a resin is provided on an outer surface of the first tube.

The handle according to one or more embodiments of the present invention allows the tube disposed in the inner cavity of the handle to be easily moved and rotated while supplying electricity to the puncture needle. The catheter system including the handle also has the same effects.

### BRIEF DESCRIPTION OF THE DRAWINGS

FIG. 1 is a side view (partial cross-sectional view) showing an example of a catheter system according to one or more embodiments of the present invention.

FIG. 2 is a partially enlarged cross-sectional view (partial side view) of the catheter system shown in FIG. 1.

FIG. 3 is a side view (partial cross-sectional view) showing a modification of the catheter system according to one or more embodiments of the present invention.

FIG. 4 is an end view of a cut section along a line IV-IV shown in FIG. 3.

FIG. 5 is an end view of a cut section showing a modification of the catheter system shown in FIG. 4.

FIG. 6 is a cross-sectional view of a puncture needle and a tube of the catheter system shown in FIG. 1.

FIG. 7 is a cross-sectional view showing a modification of the puncture needle and the tube shown in FIG. 6.

### DETAILED DESCRIPTION

One or more embodiments of the present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of one or more embodiments of the present invention.

One or more embodiments of the handle of the present invention have the gist of a handle having an inner cavity into which a tube connected to a proximal end portion of a medical puncture needle having a longitudinal direction is inserted, the tube having an inner cavity extending in the longitudinal direction of the puncture needle, wherein in a state where the tube is inserted into the inner cavity of the handle, the handle includes a contact member including a portion that is in contact with an outer surface of the tube, in a cross-section passing through a central axis of the tube and extending along a longitudinal direction of the tube, the outer surface of the tube includes a plurality of contact portions that are in contact with the contact member, and a non-contact portion that is present between the contact portions and is not in contact with the contact member, and the puncture needle and the contact member are electrically connected to each other via the contact portions.

With reference to FIG. 1 to FIG. 7, the overall configurations of a handle according to one or more embodiments of the present invention and a catheter system including the handle will be described. FIG. 1 and FIG. 3 show a catheter system 100 including a puncture needle 3, a tube 4, a sheath 5, and a handle 6. In these drawings, the longitudinal direction of the puncture needle 3 is indicated by x, the radial direction of the puncture needle 3 is indicated by y, and the circumferential direction of the puncture needle 3 is indicated by c. The radial direction y is a direction perpendicular to the longitudinal direction x. The longitudinal direction x of the puncture needle 3 can also be referred to as the extending direction of the puncture needle 3. For ease of understanding, these drawings illustrate an embodiment in which the longitudinal direction x of the puncture needle 3, the longitudinal direction of the tubular member 10, and the longitudinal direction of the tube 4 coincide with each other, the radial direction y of the puncture needle 3, the radial direction of the tubular member 10, and the radial direction of the tube 4 coincide with each other, and the circumferential direction c of the puncture needle 3, the circumferential direction of the tubular member 10, and the circumferential direction of the tube 4 coincide with each other, but the present invention is not limited to these embodiments.

In this description, a proximal side refers to the hand side of a user with respect to the longitudinal direction x of the puncture needle 3, and a distal side refers to a side opposite to the proximal side, that is, a treatment target side. In addition, when each member is divided into two equal portions in the longitudinal direction x of the puncture needle 3, the portion of the member located on the distal side is referred to as distal portion, and the portion of the member located on the proximal side is referred to as proximal portion. The distal end of each member is an end located on the most distal side of the member. The proximal end of each member is an end located on the most proximal side of the member. An end portion of each member refers to a portion of the member including its end and an area therearound. That is, a distal end portion of each member refers to a portion of the member including its distal end and an area therearound, and a proximal end portion of each member refers to a portion of the member including its proximal end and an area therearound.

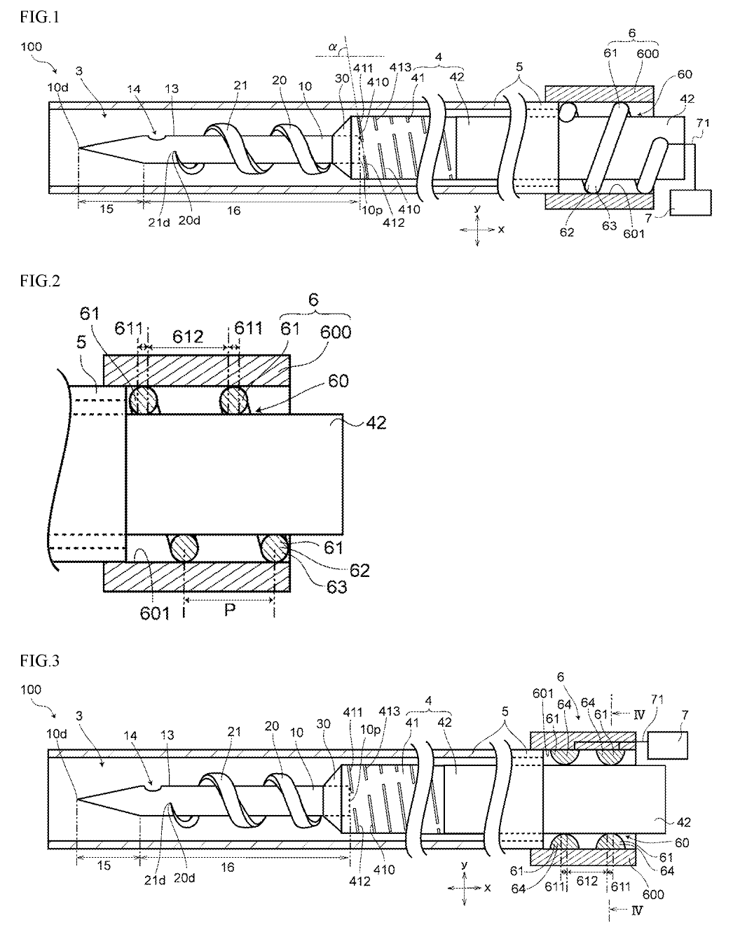

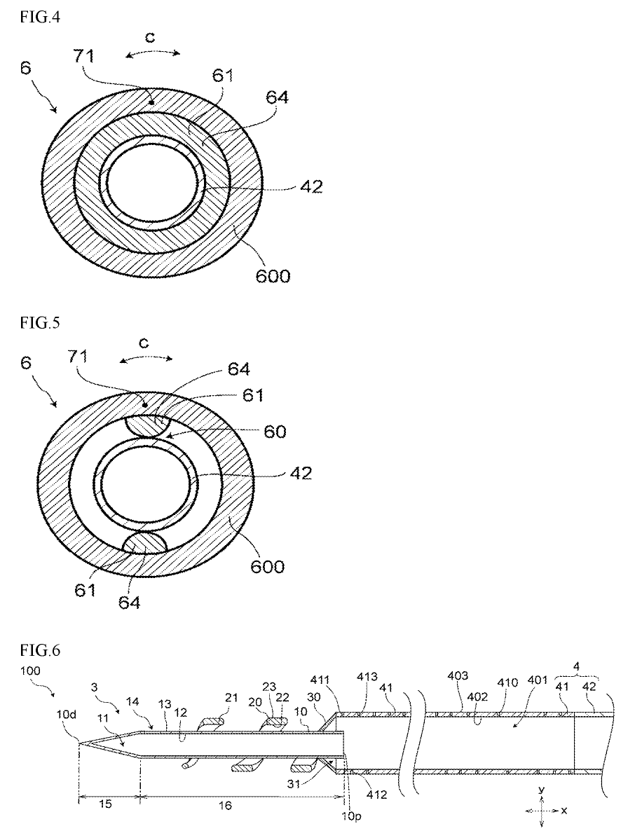

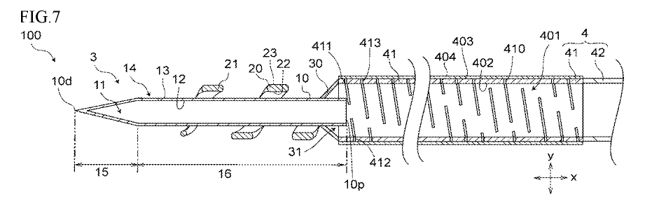

FIG. 1 is a side view (partial cross-sectional view) showing an example of the catheter system according to one or more embodiments of the present invention. More specifically, the cross-section shown in FIG. 1 is a cross-section passing through the central axis of a tube and extending along the longitudinal direction of the tube. FIG. 2 is a partially enlarged cross-sectional view (partial side view) of the catheter system shown in FIG. 1. More specifically, FIG. 2 is an enlarged cross-sectional view (partial side view) of a portion where the handle is provided. FIG. 3 is a side view (partial cross-sectional view) showing a modification of the catheter system according to one or more embodiments of the present invention. More specifically, the cross-section shown in FIG. 3 is a cross-section passing through the central axis of the tube and extending along the longitudinal direction of the tube. FIG. 4 is an end view of a cut section along a line IV-IV shown in FIG. 3. FIG. 5 is an end view of a cut section showing a modification of the catheter system shown in FIG. 4. FIG. 6 is a cross-sectional view of the puncture needle and the tube of the catheter system shown in FIG. 1. FIG. 7 is a cross-sectional view showing a modification of the puncture needle and the tube shown in FIG. 6. In FIG. 6, and FIG. 7, a sheath and the handle are not shown.

First, the handle 6 according to one or more embodiments of the present invention will be described.

As shown in FIG. 1 and FIG. 2, the handle 6 has an inner cavity 60. The tube 4 is inserted into the inner cavity 60 of the handle 6.

The puncture needle 3 is for medical use and is intended to puncture a target tissue. As shown in FIG. 1, the puncture needle 3 has the longitudinal direction x.

The tube 4 has an inner cavity extending in the longitudinal direction x of the puncture needle 3. The tube 4 is connected to a proximal end portion of the puncture needle 3.

As shown in FIG. 1 to FIG. 3, in a state where the tube 4 is inserted into the inner cavity 60 of the handle 6, the handle 6 includes a contact member 61 including a portion that is in contact with the outer surface of the tube 4. In a cross-section passing through the central axis of the tube 4 and extending along the longitudinal direction of the tube 4, the outer surface of the tube 4 includes a plurality of contact portions 611 that are in contact with the contact member 61 and a non-contact portion 612 that is present between the contact portions 611 and is not in contact with the contact member 61. The puncture needle 3 and the contact member 61 are electrically connected to each other via the contact portions 611.

Since the non-contact portion 612 is present between the plurality of contact portions 611, it becomes easier to reduce the area of contact between the outer surface of the tube 4 and the contact member 61. Therefore, it can be made easier to reduce frictional resistance generated between the outer surface of the tube 4 and the contact member 61. Accordingly, it becomes easier to move the tube 4 in the longitudinal direction x of the puncture needle 3 and to rotate the tube 4 in the circumferential direction c of the puncture needle 3 within the handle 6. In addition, since the puncture needle 3 and the contact member 61 are electrically connected to each other via the contact portions 611, electricity can be supplied to the puncture needle 3. Therefore, the handle 6 allows the tube 4 disposed in the inner cavity 60 of the handle 6 to be easily moved and rotated while supplying electricity to the puncture needle 3.

The handle 6 is a member including a portion to be gripped by the user.

The material of the handle 6 is not particularly limited, and synthetic resins including polyolefin resins such as polypropylene (PP) and polyethylene (PE), polyester resins such as polyethylene terephthalate (PET), polycarbonate resins, ABS resins, polyurethane resins, etc., can be used.

As shown in FIG. 1 to FIG. 5, the handle 6 may include a grip member 600 to be gripped by the user. It is preferable that the grip member 600 has a shape that allows the grip member 600 to be easily gripped by the user. The grip member 600 may have a shape with an inner cavity, more specifically, a hollow cylindrical shape, a hollow truncated cone shape, a hollow polygonal prism shape, a hollow truncated polygonal pyramid shape, a combination thereof, or the like. The grip member 600 can be configured to have an inner surface 601 facing the inner cavity of the grip member 600 and an outer surface facing the exterior of the grip member 600.

As shown in FIG. 1 to FIG. 5, the handle 6 includes a contact member 61 including a portion that is in contact with the outer surface of the tube 4.

The contact member 61 may be a coil 62 obtained by helically winding a wire 63. When the wire 63 is wound, the wire 63 has an inner surface located on the inner side thereof and an outer surface located on the outer side thereof. The inner cavity formed on the inner surface side of the wire 63 is the inner cavity of the coil 62. The coil 62 can be configured to have an inner surface facing the inner cavity of the coil 62 and an outer surface facing the exterior thereof. The coil 62 has a distal end and a proximal end in the longitudinal direction x of the puncture needle 3.

When the contact member 61 is the coil 62, it is preferable that the handle 6 is composed of the grip member 600 and the coil 62 disposed in the inner cavity of the grip member 600 as shown in FIG. 1 and FIG. 2. In this case, the inner cavity 60 of the handle 6 is formed by the grip member 600 and the coil 62.

The outer surface of the coil 62 and the inner surface 601 of the grip member 600 may be in contact with each other. The outer surface of the coil 62 and the inner surface 601 of the grip member 600 may be fixed to each other. The outer surface of the coil 62 and the inner surface 601 of the grip member 600 may be indirectly fixed via another member, or may be directly fixed to each other without another member being interposed therebetween. It is preferable that the coil 62 does not move relative to the grip member 600 in the longitudinal direction x of the puncture needle 3.

Direct fixation of the coil 62 and the grip member 600 refers to a state where the coil 62 and the grip member 600 are fixed to each other without another member being interposed therebetween. For example, fixation of the coil 62 and the grip member 600 by an adhesive, fixation of the coil 62 and the grip member 600 by welding, etc., correspond to the direct fixation.

Indirect fixation of the coil 62 and the grip member 600 refers to a state where the coil 62 and the grip member 600 are fixed via another member. For example, fixation of the coil 62 and the grip member 600 via a connection member that connects the coil 62 and the grip member 600 corresponds to the indirect fixation.

When the handle 6 is composed of the grip member 600 and the coil 62, it is preferable that the tube 4 is inserted into the inner cavity of the coil 62. It is preferable that the inner surface of the coil 62 and the outer surface of the tube 4 are in contact with each other but are not fixed to each other. It is preferable that the tube 4 moves relative to the coil 62 in the longitudinal direction x of the puncture needle 3 and in the circumferential direction c of the puncture needle 3.

As shown in FIG. 2, it is preferable that a pitch P of the coil 62 is larger than the wire diameter of the wire 63 of the coil 62. By adopting this configuration, it becomes easier to reduce the area of contact between the outer surface of the tube 4 and the contact member 61. Therefore, it can be made easier to reduce frictional resistance generated between the outer surface of the tube 4 and the contact member 61. Accordingly, it becomes easier to move the tube 4 in the longitudinal direction x of the puncture needle 3 and to rotate the tube 4 in the circumferential direction c of the puncture needle 3 within the handle 6.

The contact member 61 may be a projecting member 64 provided so as to project radially inward of the tube 4. When the contact member 61 is the projecting member 64, it is preferable that the handle 6 is composed of the grip member 600 and the projecting member 64 fixed to the inner surface 601 of the grip member 600 as shown in FIG. 3 to FIG. 5. In this case, the inner cavity 60 of the handle 6 is formed by the grip member 600 and the projecting member 64.

In the cross-section passing through the central axis of the tube 4 and extending along the longitudinal direction of the tube 4, the cross-sectional shape of the projecting member 64 can be a semicircular shape, a triangular shape, a polygonal shape, a combination thereof, or the like.

As shown in FIG. 3, it is preferable that a plurality of projecting members 64 are provided and that one projecting member 64 and another projecting member 64 are separated from each other. By adopting this configuration, it can be made easier to reduce the area of contact between the outer surface of the tube 4 and the contact member 61. Therefore, it can be made easier to reduce frictional resistance generated between the outer surface of the tube 4 and the contact member 61. Accordingly, it becomes easier to move the tube 4 in the longitudinal direction x of the puncture needle 3 and to rotate the tube 4 in the circumferential direction c of the puncture needle 3 within the handle 6.

As shown in FIG. 2 and FIG. 3, it is preferable that in the cross-section passing through the central axis of the tube 4 and extending along the longitudinal direction of the tube 4, the length of the contact portion 611 in the longitudinal direction x of the puncture needle 3 is shorter than the length of the non-contact portion 612 in the longitudinal direction x of the puncture needle 3. When multiple contact portions 611 exist in the cross-section passing through the central axis of the tube 4 and extending along the longitudinal direction of the tube 4, the lengths of the respective contact portions 611 in the longitudinal direction x of the puncture needle 3 are summed and regarded as the length of the contact portion 611 in the longitudinal direction x of the puncture needle 3. When multiple non-contact portions 612 exist in the cross-section passing through the central axis of the tube 4 and extending along the longitudinal direction of the tube 4, the lengths of the respective non-contact portions 612 in the longitudinal direction x of the puncture needle 3 are summed and regarded as the length of the non-contact portion 612 in the longitudinal direction x of the puncture needle 3. By adopting this configuration, it can be made easier to reduce the area of contact between the outer surface of the tube 4 and the contact member 61. Therefore, it can be made easier to reduce frictional resistance generated between the outer surface of the tube 4 and the contact member 61. Accordingly, it becomes easier to move the tube 4 in the longitudinal direction x of the puncture needle 3 and to rotate the tube 4 in the circumferential direction c of the puncture needle 3 within the handle 6.

It is preferable that the contact member 61 is made of a conductive material. The entire contact member 61 may be made of a conductive material, or only a part of the contact member 61 may be made of a conductive material. When only a part of the contact member 61 is made of a conductive material, it is preferable that the portion that is in contact with the outer surface of the tube 4 is made of a conductive material. Accordingly, it becomes easier to electrically connect the puncture needle 3 and the contact member 61 via the tube 4.

It is preferable that the contact member 61 is connected to a power supply device 7 via a conducting wire 71. Accordingly, electricity can be supplied to the puncture needle 3 via the contact member 61 and the tube 4.

Next, the catheter system according to one or more embodiments of the present invention will be described.

As shown in FIG. 1 to FIG. 3, the catheter system 100 includes the handle 6 having any of the above-described configurations.

As shown in FIG. 1, FIG. 3, FIG. 6, and FIG. 7, it is preferable that the puncture needle 3 included in the catheter system 100 includes: the tubular member 10 having the inner cavity 11 extending in the longitudinal direction x of the puncture needle 3; and the helical member 20 in which the wire 21 is wound helically around the tubular member 10. Since the helical member 20 is disposed around the tubular member 10 in the puncture needle 3, the puncture needle 3 can be easily screwed into the target tissue by rotating the puncture needle 3 about the tubular member 10 as an axis. Accordingly, it becomes easier for the puncture needle 3 to puncture the target tissue. In addition, the wire 21 constituting the helical member 20 bites into the tissue, so that it becomes difficult for the puncture needle 3 to come out of the tissue. Furthermore, the puncture depth of the puncture needle 3 can be easily adjusted by adjusting the rotation of the puncture needle 3.

As shown in FIG. 1, FIG. 3, FIG. 6, and FIG. 7, the catheter system 100 includes the puncture needle 3.

The puncture needle 3 is used when administering a liquid such as a cell preparation or a drug solution to the target tissue. Specifically, the puncture needle 3 can be used when directly administering such a liquid to an organ in the body such as the heart, kidneys, or liver. For example, the puncture needle 3 can be used when directly administering an iPS cell suspension to the liver or kidneys, or when directly administering a myocardial regenerative cell preparation to the heart, more specifically to the cardiac muscle.

It is preferable that the puncture needle 3 is intended to puncture organs in the body. The organs in the body refer to organs within the body, particularly those located in the abdominal or thoracic regions, and are portions commonly referred to as viscera.

The puncture needle 3 can be made of a metal or resin, for example. The entire puncture needle 3 may be made of only a metal or may be made of only a resin. A part of the puncture needle 3 may be made of a metal, and the other portion of the puncture needle 3 may be made of a resin. The tubular member 10 and the helical member 20 may be made of the same material or may be made of different materials.

It is preferable that the puncture needle 3 is made of only a metal. Examples of the metal forming the puncture needle 3 include stainless steels such as SUS304 and SUS316, platinum, nickel, cobalt, chromium, titanium, tungsten, gold, Ni-Ti alloys, Co-Cr alloys, and combinations thereof.

Examples of the resin forming the puncture needle 3 include polyether ether ketone (PEEK) and polycarbonate (PC). When the puncture needle 3 is made of only a resin without using any metal, the puncture needle 3 can be used for patients with metal allergies.

The length of the puncture needle 3 in the longitudinal direction x of the puncture needle 3 can be set to 2 mm or more, 3 mm or more, 4 mm or more, etc. The length of the puncture needle 3 in the longitudinal direction x of the puncture needle 3 can be set to 50 mm or less, 30 mm or less, 10 mm or less, etc. The length of the puncture needle 3 in the longitudinal direction x of the puncture needle 3 refers to the maximum length among lengths of the puncture needle 3 in the longitudinal direction x of the puncture needle 3. When the puncture needle 3 is used as a puncture needle for puncturing the cardiac muscle, it is preferable that the length of the puncture needle 3 in the longitudinal direction x of the puncture needle 3 is 5 mm.

The length of the puncture needle 3 in the radial direction y of the puncture needle 3 can be set to 0.2 mm or more, 0.3 mm or more, 0.4 mm or more, etc. The length of the puncture needle 3 in the radial direction y of the puncture needle 3 can be set to 10 mm or less, 5 mm or less, 1 mm or less, etc. The length of the puncture needle 3 in the radial direction y of the puncture needle 3 refers to the maximum length among lengths of the puncture needle 3 in the radial direction y of the puncture needle 3. When the puncture needle 3 is used as a puncture needle for puncturing the cardiac muscle, it is preferable that the length of the puncture needle 3 in the radial direction y of the puncture needle 3 is 0.45 mm.

As shown in FIG. 1, FIG. 3, FIG. 6, and FIG. 7, the tubular member 10 is a member having a tubular shape and has the inner cavity 11 extending in the longitudinal direction x of the puncture needle 3. A liquid such as a cell preparation or a drug solution can be delivered to the target tissue through the inner cavity 11.

The tubular member 10 can be configured to have an inner surface 12 facing the inner cavity 11 and the outer surface 13 facing the exterior thereof. The tubular member 10 has a distal end 10d and a proximal end 10p in the longitudinal direction x of the puncture needle 3. As for the tubular member 10, it is preferable that the distal end 10d is closed and the proximal end 10p is open.

As shown in FIG. 1, it is preferable that the tubular member 10 includes: a tapered portion 15 having an outer diameter decreasing toward the distal side; and a straight tube portion 16 located on the proximal side with respect to the tapered portion 15. In particular, it is preferable that the tapered portion 15 is provided at a portion including the distal end 10d of the tubular member 10. Accordingly, it is made possible for a distal end portion of the tubular member 10 to puncture the target tissue. In addition, when the puncture needle 3 is rotated in the circumferential direction c of the puncture needle 3, the deviation of the rotation axis thereof is reduced, thereby making it easier to improve stability during puncturing with the puncture needle 3.

As shown in FIG. 1, FIG. 3, FIG. 6, and FIG. 7, it is preferable that a hole 14 providing communication between the inner cavity 11 of the tubular member 10 and the exterior of the puncture needle 3 is formed in the tubular member 10. A liquid such as a cell preparation or a drug solution delivered through the inner cavity 11 is delivered to the target tissue through the hole 14.

It is preferable that the hole 14 is a hole through which the liquid can be discharged from the inner cavity 11 of the tubular member 10 toward the radially outer side of the tubular member 10. Therefore, it is preferable that the hole 14 is located on the proximal side with respect to the distal end 10d of the tubular member 10 and on the distal side with respect to the proximal end 10p of the tubular member 10.

Only one hole 14 may be formed in the tubular member 10, or a plurality of holes 14 may be formed in the tubular member 10. When only one hole 14 is formed in the tubular member 10, it becomes easier to administer a liquid such as a cell preparation or a drug solution in a pinpoint manner. When a plurality of holes 14 are formed in the tubular member 10, it becomes easier to administer a liquid such as a cell preparation or a drug solution over a wide area.

The outer shape of the hole 14 when observed in a direction perpendicular to the longitudinal direction x of the puncture needle 3 can be a polygonal shape such as a triangular shape, a quadrilateral shape, or a pentagonal shape, a circular shape, an elliptical shape, or a combination thereof. The polygonal shape includes not only a shape in which the vertex of each corner portion is clearly defined and each side is straight but also a polygonal shape with curved corners and a shape in which at least one or some of the sides are curved.

As shown in FIG. 1, FIG. 3, FIG. 6, and FIG. 7, the puncture needle 3 includes the helical member 20 in which the wire 21 is wound helically around the tubular member 10.

The helical member 20 is formed by helically winding the wire 21. When the wire 21 is wound, the wire 21 has an inner surface located on the inner side thereof and an outer surface located on the outer side thereof. The inner cavity formed on the inner surface side of the wire 21 is the inner cavity of the helical member 20. The tubular member 10 is disposed in the inner cavity of the helical member 20. The helical member 20 can be configured to have an inner surface 22 facing the inner cavity of the helical member 20 and an outer surface 23 facing the exterior thereof. The helical member 20 has a distal end 20d and a proximal end in the longitudinal direction x of the puncture needle 3.

Only one helical member 20 may be provided in the puncture needle 3, or a plurality of helical members 20 may be provided in the puncture needle 3.

The cross-sectional shape of the wire 21 in the cross-section perpendicular to the longitudinal direction x of the puncture needle 3 can be a polygonal shape such as a triangular shape, a quadrilateral shape, or a pentagonal shape, a circular shape, an elliptical shape, or a combination thereof. In the cross-section perpendicular to the longitudinal direction x of the puncture needle 3, the cross-sectional shape of the wire 21 may be a polygonal shape or may be a quadrilateral shape. The cross-sectional shape of the wire 21 in the cross-section perpendicular to the longitudinal direction x of the puncture needle 3 may be the same from the distal end 21d to the proximal end of the wire 21. When the cross-sectional shape of the wire 21 in the cross-section perpendicular to the longitudinal direction x of the puncture needle 3 is the same from the distal end 21d to the proximal end of the wire 21, the size of the cross-sectional shape may be the same (congruent) or different (similar) depending on the position in the longitudinal direction x of the puncture needle 3.

The cross-sectional shape of the wire 21 in the cross-section perpendicular to the longitudinal direction x of the puncture needle 3 may be different depending on the position in the longitudinal direction x of the puncture needle 3. For example, the cross-sectional shape of the wire 21 in a cross-section perpendicular to the longitudinal direction x of the puncture needle 3 and passing through the midpoint of the puncture needle 3 in the longitudinal direction x is a rectangular shape, and the cross-sectional shape of the wire 21 in a cross-section perpendicular to the longitudinal direction x of the puncture needle 3 and passing through the distal end portion of the wire 21 may be an elliptical shape. Alternatively, the cross-sectional shape of the wire 21 in the cross-section perpendicular to the longitudinal direction x of the puncture needle 3 and passing through the midpoint of the puncture needle 3 in the longitudinal direction x may be a rectangular shape in which two long sides have an arc shape.

As shown in FIG. 6 and FIG. 7, it is preferable that the wire 21 has a solid structure. The entire wire 21 may have a solid structure, or only a part of the wire 21 may have a solid structure. In particular, it is preferable that the entire distal end portion of the wire 21 has a solid structure. When the wire 21 is made to have a solid structure, it becomes easier to reduce the diameter of the wire 21. However, although not illustrated, an embodiment in which the wire 21 has a hollow structure is also permissible. For example, the wire 21 may have an inner cavity formed to extend along the central axis of the wire 21.

The wire 21 may be a single wire or a stranded wire.

It is preferable that the distal end 20d of the helical member 20 is located on the proximal side with respect to the distal end 10d of the tubular member 10. The distal end 20d of the helical member 20 may be located on the distal side with respect to the proximal end of the tapered portion 15 of the tubular member 10, but it is preferable that the distal end 20d of the helical member 20 is located on the proximal side with respect to the proximal end of the tapered portion 15 of the tubular member 10. The wire 21 may be disposed only radially outward of the straight tube portion 16 of the tubular member 10 and may not necessarily be disposed radially outward of the tapered portion 15 of the tubular member 10. Owing to this configuration, the distal end portion of the tubular member 10 can be brought into contact with the target tissue before a distal end portion of the helical member 20. Accordingly, it can be made easier to rotate the puncture needle 3 about the tubular member 10 as an axis when rotating the puncture needle 3 in the circumferential direction c of the puncture needle 3, so that the deviation of the rotation axis thereof is easily reduced, thereby making it easier to improve stability during puncturing with the puncture needle 3.

A part of the tubular member 10 and a part of the helical member 20 may be fixed to each other. More specifically, it is preferable that a proximal portion of the helical member 20 is fixed to a proximal portion of the tubular member 10, or it is preferable that a proximal end portion of the helical member 20 is fixed to the proximal portion of the tubular member 10. Accordingly, it becomes easier to stably screw the puncture needle 3 into the tissue without the position of the helical member 20 being changed relative to the tubular member 10. The tubular member 10 and the helical member 20 may be indirectly fixed via another member, but it is preferable that a part of the tubular member 10 and a part of the helical member 20 are directly fixed to each other without another member being interposed therebetween.

Direct fixation of the tubular member 10 and the helical member 20 refers to a state where the tubular member 10 and the helical member 20 are fixed to each other without another member being interposed therebetween. For example, fixation of the tubular member 10 and the helical member 20 by an adhesive, fixation of the tubular member 10 and the helical member 20 by welding, etc., correspond to the direct fixation.

Indirect fixation of the tubular member 10 and the helical member 20 refers to a state where the tubular member 10 and the helical member 20 are fixed via another member. For example, fixation of the tubular member 10 and the helical member 20 via a connection member that connects the outer surface 13 of the tubular member 10 and the inner surface 22 of the helical member 20 corresponds to the indirect fixation.

The proximal end portion of the helical member 20 refers to a portion of the helical member 20 including its proximal end and an area therearound. The range of the proximal end portion of the helical member 20 may be, for example, the following range. The range of the proximal end portion of the helical member 20 may be a range from the proximal end of the helical member 20 to a point located on the distal side from the proximal end of the helical member 20 at a distance that is 1/3 of the length of the helical member 20 when the helical member 20 is divided into three equal portions in the longitudinal direction x of the puncture needle 3. The range of the proximal end portion of the helical member 20 may be a range from the proximal end of the helical member 20 to a point located on the distal side from the proximal end of the helical member 20 at a distance that is 1/4 of the length of the helical member 20 when the helical member 20 is divided into four equal portions in the longitudinal direction x of the puncture needle 3. The range of the proximal end portion of the helical member 20 may be a range from the proximal end of the helical member 20 to a point located on the distal side from the proximal end of the helical member 20 at a distance that is 1/5 of the length of the helical member 20 when the helical member 20 is divided into five equal portions in the longitudinal direction x of the puncture needle 3.

The inner diameter of the helical member 20 may be four times or less or three times or less the outer diameter of the tubular member 10, but may be two times or less the outer diameter of the tubular member 10. By adopting this configuration, the gap between the tubular member 10 and the helical member 20 tends to be smaller, thereby making it easier to screw the puncture needle 3 into the target tissue by rotating the puncture needle 3 about the tubular member 10 as an axis. It is preferable that the inner diameter of the helical member 20 is larger than the outer diameter of the tubular member 10, and the inner diameter of the helical member 20 can be set to 1.1 times or more, 1.2 times or more, 1.3 times or more, etc., the outer diameter of the tubular member 10.

It is preferable that, in the cross-section perpendicular to the longitudinal direction x of the puncture needle 3 and passing through the midpoint of the puncture needle 3 in the longitudinal direction x, the length of the wire 21 in the radial direction of the tubular member 10 is shorter than the length of the wire 21 in the circumferential direction of the tubular member 10. The longer the length of the wire 21 in the radial direction of the tubular member 10 is, the easier it becomes to adjust the puncture depth; however, in some cases, it may become more difficult for the puncture needle 3 to puncture the tissue, requiring more time for puncturing. By adopting the above configuration, the length of the wire 21 in the radial direction of the tubular member 10 can be made relatively short, so that it becomes easier to adjust the puncture depth without making it excessively difficult for the puncture needle 3 to puncture the tissue. The length of the wire 21 in the radial direction of the tubular member 10 refers to the maximum length among lengths of the wire 21 in the radial direction of the tubular member 10. The length of the wire 21 in the circumferential direction of the tubular member 10 refers to the maximum length among lengths of the wire 21 in the circumferential direction of the tubular member 10.

As shown in FIG. 1, it is preferable that the wire 21 has a tapered shape extending from the distal end 21d of the wire 21 to a position away from the distal end 21d of the wire 21 toward the proximal side by a length equal to 1/2 of a pitch P of the helical member 20. By having a tapered shape extending to the above position, it becomes easier for the wire 21 to enter the target tissue, so that it becomes easier to screw the puncture needle 3 into the target tissue. The tapered shape may be a shape that becomes narrower toward the distal end 21d of the wire 21.

As shown in FIG. 1, FIG. 3, FIG. 6, and FIG. 7, the catheter system 100 includes the tube 4 to which the puncture needle 3 is connected. It is preferable that the tube 4 is connected to the proximal end portion of the puncture needle 3 and has an inner cavity extending in the longitudinal direction x of the puncture needle 3. It is preferable that the proximal end portion of the puncture needle 3 is connected to a distal end portion of the tube 4.

The tube 4 can be made of, for example, any of the materials exemplified as those that can form the puncture needle 3.

The tube 4 may be composed of only one member having a tubular shape, or may be composed of a plurality of members having a tubular shape.

As shown in FIG. 1, FIG. 3, FIG. 6, and FIG. 7, it is preferable that the tube 4 includes a first tube 41 in which a plurality of grooves 410 are formed. When the plurality of grooves 410 are formed in the first tube 41, flexibility can be imparted to the first tube 41. In addition, when the tube 4 including the first tube 41 in which the grooves 410 are formed is connected to the puncture needle 3, it can be made easier to transmit torque to the puncture needle 3. Therefore, the flexibility and operability of the catheter system 100 can be improved.

As shown in FIG. 6 and FIG. 7, the first tube 41 can be configured to have an inner cavity 401 extending in the longitudinal direction x of the puncture needle 3, an inner surface 402 facing the inner cavity 401, and an outer surface 403 facing the exterior thereof. The first tube 41 has a distal end and a proximal end in the longitudinal direction x of the puncture needle 3. As for the first tube 41, it is preferable that both the distal end and the proximal end thereof are open.

The shape of the first tube 41 can be, for example, a hollow cylindrical shape, a hollow polygonal prism shape, or the like.

The length of the first tube 41 in the longitudinal direction x of the puncture needle 3 can be set to 100 mm or more, 130 mm or more, 160 mm or more, etc. The length of the first tube 41 in the longitudinal direction x of the puncture needle 3 can be set to 300 mm or less, 270 mm or less, 240 mm or less, etc. The length of the first tube 41 in the longitudinal direction x of the puncture needle 3 refers to the maximum length among lengths of the first tube 41 in the longitudinal direction x of the puncture needle 3. When used as a catheter system for puncturing the cardiac muscle, it is preferable that the length of the first tube 41 in the longitudinal direction x of the puncture needle 3 is 200 mm.

The length of the first tube 41 in the radial direction y of the puncture needle 3 can be set to 0.3 mm or more, 0.5 mm or more, 0.8 mm or more, etc. The length of the first tube 41 in the radial direction y of the puncture needle 3 can be set to 20 mm or less, 15 mm or less, 10 mm or less, etc. The length of the first tube 41 in the radial direction y of the puncture needle 3 refers to the maximum length among lengths of the first tube 41 in the radial direction y of the puncture needle 3. When used as a catheter system for puncturing the cardiac muscle, it is preferable that the length of the first tube 41 in the radial direction y of the puncture needle 3 is 0.9 mm.

As shown in FIG. 6 and FIG. 7, it is preferable that the inner cavity 11 of the tubular member 10 communicates with the inner cavity 401 of the first tube 41. Accordingly, a liquid such as a cell preparation or a drug solution can be delivered to the puncture needle 3 through the inner cavity 401 of the first tube 41.

The first tube 41 can be made of, for example, any of the materials exemplified as those that can form the puncture needle 3, but it is preferable that the first tube 41 is made of a metal.

The grooves 410 formed in the first tube 41 may be through grooves as shown in FIG. 7, or may be bottomed grooves as shown in FIG. 6. Both through grooves and bottomed grooves may be formed in the first tube 41. When through grooves and bottomed grooves are combined or either through grooves or bottomed grooves are selected for the grooves 410 formed in the first tube 41, it becomes easier to provide the first tube 41 with desired flexibility and rigidity.

As shown in FIG. 7, it is preferable that a first resin layer 404 made of a resin is provided on the outer surface 403 of the first tube 41. Accordingly, regardless of whether the grooves 410 formed in the first tube 41 are through grooves or bottomed grooves, a liquid such as a cell preparation or a drug solution can be delivered to the puncture needle 3 through the inner cavity 401 of the first tube 41. When grooves 410 are through grooves, it is preferable that the first resin layer 404 is provided on the outer surface 403 of the first tube 41.

The first resin layer 404 may be provided so as to cover the entire outer surface 403 of the first tube 41, or may be provided so as to cover only a part of the outer surface 403 of the first tube 41. As shown in FIG. 3, the first resin layer 404 may be provided over the grooves 410.

The first resin layer 404 can be made of, for example, a synthetic resin such as a polyolefin resin (e.g., polyethylene or polypropylene), a polyamide resin (e.g., nylon), a polyester resin (e.g., PET), an aromatic polyether ketone resin (e.g., PEEK), a polyether polyamide resin, a polyurethane resin, a polyimide resin, or a fluorine resin (e.g., PTFE, PFA, or ETFE).

As shown in FIG. 1, it is preferable that when observed in the direction perpendicular to the longitudinal direction x of the puncture needle 3, the extending directions of the grooves 410 and the longitudinal direction x of the puncture needle 3 intersect at an angle α. Each angle α at which the extending direction of each groove 410 and the longitudinal direction x of the puncture needle 3 intersect may be the same or different. The angle α may be 70° or more, 73° or more, or 75° or more. The angle α may be 85° or less, 83° or less, or 80° or less. When the angle α is set within the above range, it can be made easier to transmit torque to the puncture needle 3.

It is preferable that the length of one groove 410 in the extending direction of the groove 410 is shorter than the length of the outer circumference of the first tube 41. The length of one groove 410 in the extending direction of the groove 410 refers to the maximum length among lengths of one groove 410 in the extending direction of the groove 410.

As shown in FIG. 1, it is preferable that the plurality of grooves 410 include a first groove 411 and a second groove 412 located at a position different from that of the first groove 411, and that, when observed in the direction perpendicular to the longitudinal direction x of the puncture needle 3, the second groove 412 is located in the extending direction of the first groove 411. Accordingly, it can be made easier to transmit torque to the puncture needle 3.

As shown in FIG. 1, it is preferable that the maximum length between the first groove 411 and the second groove 412 in the extending direction of the first groove 411 is shorter than the maximum length of the first groove 411 in the extending direction of the first groove 411. Accordingly, the interval between the first groove 411 and the second groove 412 can be made relatively short, thereby making it easier to impart flexibility to the first tube 41.

As shown in FIG. 1, it is preferable that the plurality of grooves 410 include a third groove 413 located adjacent to the first groove 411 in the longitudinal direction x of the puncture needle 3, and that the maximum length between the first groove 411 and the third groove 413 in the longitudinal direction x of the puncture needle 3 is shorter than the length of the pitch of the helical member 20. Accordingly, the interval between the first groove 411 and the third groove 413 can be made relatively short, thereby making it easier to impart flexibility to the first tube 41.

It is preferable that the distal end portion of the first tube 41 is connected to the puncture needle 3. The first tube 41 and the puncture needle 3 may be connected via a diameter-reducing member 30. The diameter-reducing member 30 is a member having an outer diameter decreasing toward the distal side. More specifically, as shown in FIG. 1, FIG. 3, FIG. 6, and FIG. 7, it is preferable that a proximal end portion of the tubular member 10 and the distal end portion of the first tube 41 are connected by the diameter-reducing member 30.

It is preferable that the diameter-reducing member 30 has an inner cavity 31 penetrating in the longitudinal direction x of the puncture needle 3. The diameter-reducing member 30 can be configured to have an inner surface facing the inner cavity 31 and an outer surface facing the exterior thereof. The diameter-reducing member 30 has a distal end and a proximal end in the longitudinal direction x of the puncture needle 3. As for the diameter-reducing member 30, it is preferable that both the distal end and the proximal end thereof are open. It is preferable that the inner cavity 31 of the diameter-reducing member 30 communicates with the inner cavity 11 of the tubular member 10 and the inner cavity 401 of the first tube 41.

The diameter-reducing member 30 can be made of, for example, any of the materials exemplified as those that can form the puncture needle 3, but it is preferable that the diameter-reducing member 30 is made of a metal. The material forming the first tube 41 and the material forming the diameter-reducing member 30 may be the same or different.

The shape of the diameter-reducing member 30 can be, for example, a hollow truncated polygonal pyramid shape, a hollow truncated cone shape, or the like.

As shown in FIG. 1, FIG. 3, FIG. 6, and FIG. 7, it is preferable that the tube 4 includes a second tube 42 connected to a proximal end portion of the first tube 41, and that the contact member 61 is in contact with the outer surface of the second tube 42.

The second tube 42 can be configured to have an inner cavity extending in the longitudinal direction x of the puncture needle 3, an inner surface facing the inner cavity, and an outer surface facing the exterior thereof. The second tube 42 has a distal end and a proximal end in the longitudinal direction x of the puncture needle 3. As for the second tube 42, it is preferable that both the distal end and the proximal end thereof are open.

The shape of the second tube 42 can be, for example, a hollow cylindrical shape, a hollow polygonal prism shape, or the like.

The length of the second tube 42 in the longitudinal direction x of the puncture needle 3 can be set to 1000 mm or more, 1200 mm or more, 1400 mm or more, etc. The length of the second tube 42 in the longitudinal direction x of the puncture needle 3 can be set to 2000 mm or less, 1800 mm or less, 1600 mm or less, etc. The length of the second tube 42 in the longitudinal direction x of the puncture needle 3 refers to the maximum length among lengths of the second tube 42 in the longitudinal direction x of the puncture needle 3. When used as a catheter system for puncturing the cardiac muscle, it is preferable that the length of the second tube 42 in the longitudinal direction x of the puncture needle 3 is 1300 mm.

The length of the second tube 42 in the radial direction y of the puncture needle 3 can be set to 0.3 mm or more, 0.5 mm or more, 0.8 mm or more, etc. The length of the second tube 42 in the radial direction y of the puncture needle 3 can be set to 20 mm or less, 15 mm or less, 10 mm or less, etc. The length of the second tube 42 in the radial direction y of the puncture needle 3 refers to the maximum length among lengths of the second tube 42 in the radial direction y of the puncture needle 3. When used as a catheter system for puncturing the cardiac muscle, it is preferable that the length of the second tube 42 in the radial direction y of the puncture needle 3 is 0.9 mm.

It is preferable that a distal end portion of the second tube 42 is connected to the proximal end portion of the first tube 41. It is also preferable that the inner cavity of the second tube 42 communicates with the inner cavity 401 of the first tube 41. Accordingly, a liquid such as a cell preparation or a drug solution can be delivered to the puncture needle 3 through the inner cavity 401 of the first tube 41 and the inner cavity of the second tube 42.

The second tube 42 can be made of, for example, any of the materials exemplified as those that can form the puncture needle 3, but it is preferable that the second tube 42 is made of a metal. The material forming the first tube 41 and the material forming the second tube 42 may be the same or different.

As shown in FIG. 1 and FIG. 3, it is preferable that the catheter system 100 includes the sheath 5 that has a proximal end portion connected to the handle 6 and has an inner cavity into which the puncture needle 3 and the tube 4 are inserted. It is preferable that the puncture needle 3 and the tube 4 are movable in the longitudinal direction x of the puncture needle 3 within the inner cavity of the sheath 5.

Since the sheath 5 is inserted into the body, the sheath 5 may have flexibility. Accordingly, the sheath 5 can be deformed along the shape of the body cavity. In addition, for shape retention, it is preferable that the sheath 5 has elasticity.

Examples of the sheath 5 include: a hollow body formed by arranging one or more wires in a predetermined pattern; a body obtained by coating at least one of the inner and outer surfaces of the hollow body with a resin; a resin tube; and combinations of these such as these connected in the longitudinal direction. Examples of a hollow body with a wire(s) arranged in a predetermined pattern include a tubular body having a mesh structure formed by wires simply crossed or braided, and a coil formed by winding a wire. The wire(s) may be one or more single wires or one or more stranded wires. The resin tube can be manufactured, for example, by extrusion molding. When the sheath 5 is a resin tube, the sheath 5 can be composed of a single layer or multiple layers. A part of the sheath 5 in the longitudinal direction or circumferential direction thereof may be composed of a single layer, and the other part thereof may be composed of multiple layers.

The sheath 5 can be made of, for example, a synthetic resin such as a polyolefin resin (e.g., polyethylene or polypropylene), a polyamide resin (e.g., nylon), a polyester resin (e.g., PET), an aromatic polyether ketone resin (e.g., PEEK), a polyether polyamide resin, a polyurethane resin, a polyimide resin, or a fluorine resin (e.g., PTFE, PFA, or ETFE), or a metal such as stainless steel, carbon steel, or a nickel-titanium alloy. Only one of these materials may be used alone, or two or more of these materials may be used in combination.

As shown in FIG. 1, it is preferable that the puncture needle 3 is transported to the target organ in the body in a state where the puncture needle 3 is disposed in the inner cavity of the sheath 5. After transportation, it is preferable to move the position of the puncture needle 3 relative to the sheath 5 toward the distal side and cause the puncture needle 3 to protrude from a distal end portion of the sheath 5 to puncture the organ.

It is preferable that the puncture needle 3 and the tube 4 are made of a conductive material. Accordingly, electricity can be supplied to the puncture needle 3 via the tube 4. A counter electrode used when passing electricity may be an electrode to be placed on the body surface of a patient or an electrode disposed on the outer surface of the sheath 5. As the electrode to be placed on the body surface of a patient, for example, a plate-shaped, sheet-shaped, or pad-shaped electrode can be used. As the electrode disposed on the outer surface of the sheath 5, it is preferable to use an annular, tubular, or ring-shaped electrode.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

### DESCRIPTION OF THE REFERENCE CHARACTERS

10: tubular member

10d: distal end of tubular member

10p: proximal end of tubular member

11: inner cavity of tubular member

12: inner surface of tubular member

13: outer surface of tubular member

14: hole

15: tapered portion

16: straight tube portion

20: helical member

20d: distal end of helical member

21: wire

21d: distal end of wire

22: inner surface of helical member

23: outer surface of helical member

3: puncture needle

30: diameter-reducing member

31: inner cavity of diameter-reducing member

4: tube

41: first tube

401: inner cavity of first tube

402: inner surface of first tube

403: outer surface of first tube

404: first resin layer

410: groove

411: first groove

412: second groove

413: third groove

42: second tube

5: sheath

6: handle

60: inner cavity of handle

600: grip member

601: inner surface of grip member

61: contact member

611: contact portion

612: non-contact portion

62: coil

P: pitch of coil

63: wire

64: projecting member

7: power supply device

71: conducting wire

100: catheter system

x: longitudinal direction of puncture needle

y: radial direction of puncture needle

c: circumferential direction of puncture needle

1. A handle having an inner cavity configured to receive a tube connected to a proximal end portion of a medical puncture needle having a longitudinal direction, the tube having an inner cavity extending in the longitudinal direction, wherein in a state where the tube is inserted into the inner cavity of the handle,
the handle comprises a contact member including a portion that is in contact with an outer surface of the tube,
in a cross-section passing through a central axis of the tube and extending along a longitudinal direction of the tube, the outer surface of the tube includes a plurality of contact portions that are in contact with the contact member, and a non-contact portion that is present between the contact portions and is not in contact with the contact member, and
the puncture needle and the contact member are electrically connected to each other via the contact portions.

2. The handle according to claim 1, wherein, in the cross-section passing through the central axis of the tube and extending along the longitudinal direction of the tube, a length of the contact portion in the longitudinal direction of the puncture needle is shorter than a length of the non-contact portion in the longitudinal direction of the puncture needle.

3. The handle according to claim 1, wherein the contact member is a coil in which a wire is wound helically.

4. The handle according to claim 3, wherein a pitch of the coil is larger than a wire diameter of the wire.

5. The handle according to claim 1, wherein the contact member is a projecting member provided so as to project radially inward of the tube.

6. The handle according to claim 5, wherein:
a plurality of the projecting members are provided, and
the projecting members are separated from each other.

7. The handle according to claim 1, wherein the contact member is made of a conductive material.

8. A catheter system comprising:
the handle according to claim 1;
the puncture needle;
the tube to which the puncture needle is connected; and
a sheath connected at a proximal end portion thereof to the handle and having an inner cavity in which the puncture needle and the tube are inserted.

9. The catheter system according to claim 8, wherein the puncture needle and the tube are made of a conductive material.

10. The catheter system according to claim 8, wherein the puncture needle includes: a tubular member having an inner cavity extending in the longitudinal direction of the puncture needle; and a helical member in which a wire is wound helically around the tubular member.

11. The catheter system according to claim 8, wherein the tube includes a first tube having a plurality of grooves formed therein.

12. The catheter system according to claim 11, wherein
the tube includes a second tube connected to a proximal end portion of the first tube, and
the contact member is in contact with an outer surface of the second tube.

13. The catheter system according to claim 12, wherein a first resin layer made of a resin is provided on an outer surface of the first tube.
